(19) United States
(12) Patent Application Publication
(10) Pub. No.: US 2018/0243104 A1
(43) Pub. Date: Aug. 30, 2018

(54) MODULAR INTERBODY FUSION SYSTEMS AND METHODS

(71) Applicant: G6 Solutions, LLC, Baltimore, MD (US)

(72) Inventors: Ira Garonzik, Pikesville, MD (US); James Conway, Towson, MD (US); David Skinlo, Salt Lake City, UT (US)

(73) Assignee: G6 Solutions, LLC, Baltimore, MD (US)

(21) Appl. No.: 15/879,059

(22) Filed: Jan. 24, 2018

Related U.S. Application Data
(60) Provisional application No. 62/449,859, filed on Jan. 24, 2017.

(51) Int. Cl. A61F 2/44

(52) U.S. Cl. CPC A61F 2/447; A61F 2002/30403; A61F 2002/448

(57) ABSTRACT

Interbody fusion systems and methods are provided. According to one embodiment, an interbody fusion system may include an interbody fusion device implantable in an interbody space of a spinal joint. The interbody fusion device may include a first component and a second component. The first component and the second component, together, may define a mating interface at which the first component and the second component are securable together with the first component and the second component both positioned entirely within the interbody space. The first component and the second component, together, may have at least a first cavity that is exposed on a superior side and on an inferior side of the interbody fusion device. The system may further include a first inserter securable to a first proximal end of the first component and a second inserter securable to a second proximal end of the second component.

### CROSS-REFERENCE TO RELATED APPLICATIONS

This application claims the benefit of U.S. Provisional Patent Application Ser. No. 62/449,859, filed Jan. 24, 2017 and entitled MODULAR INTERBODY FUSION SYSTEM AND METHOD (Attorney's Docket No. G6S-1PROV), which is incorporated by reference as though set forth herein in its entirety.

### TECHNICAL FIELD

The present disclosure relates to orthopedic implants, instruments, and methods, and more particularly to interbody fusion cages and methods and instruments for surgically implanting interbody fusion cages.

### BACKGROUND

Spinal fusion surgery is a common procedure to reduce or eliminate pain from nerve impingement due to the narrowing of the spinal canal. The spinal fusion procedure may include the removal of a portion of the damaged disk. The disk can be accessed via various approaches, including, for example, anterior, transverse-lateral, and postero-lateral approaches. A transverse-lateral interbody fusion, or TLIF, has the advantages of being single-sided and nerve-sparing in its approach. This approach is depicted in FIG. 1.

FIG. 1 is a perspective view depicting the transverse-lateral insertion of an interbody fusion device or device 100, also known as a “fusion cage,” according to one embodiment. The device 100 may be inserted into the interbody space 110 between the end plate of a first vertebral body 120 and the corresponding end plate of a second, adjacent vertebral body (not shown). An inserter 130 may be used to insert the device 100 through an opening 140 in the periphery 150 of the natural disc. As shown, much of the periphery 150 is saved by this approach; preservation of the periphery 150 may help maintain bone graft position within the interbody space 110 and expedite the healing process.

After insertion, the device 100 may help improve overall joint spacing and relieve the nerve pressure. The device 100 may be constructed from any of a wide variety of materials. For example, PEEK (polyaryletherketone), Titanium and/or various ceramics may be used depending on factors such as the design of the device 100, the access to the interbody space 110 available for the device 100, the desired healing characteristics, and/or the need for radiological transparency or opacity. As shown, the device 100 may have a cavity 160 in which a quantity of bone graft or other material may be placed to help encourage bone growth between the end plates of the adjacent vertebral bodies.

FIG. 2 depicts a device 200 according to another embodiment. The device 200 has a larger “footprint,” or size in the medial-lateral and anterior-posterior directions, than the device 100. Accordingly, by comparison with the device 100, the device 200 may provide additional stability, but at the cost of more stringent access requirements and possibly more disruptive insertion.

In addition to insertion of the device 100 or the device 200, pedicle screws and rods may be added to provide additional structural support to the fused joint and/or aid in the healing process of the joint. This will be shown in connection with FIGS. 3A and 3B.

FIGS. 3A and 3B depict posterior elevation and side elevation views, respectively, of a system 360 including an interbody fusion device, or device 300, and rods 320 retained by pedicle screws 310 anchored in the pedicles of the vertebrae 330. As shown, the rods 320 may span the joint being fused to help maintain the space and reduce motion while the fused joint heals.

The likelihood of a successful spinal fusion procedure may be increased by using “tissue saving” implants and/or methods. Reducing the amount of tissue damage is a key concern for surgeons; as such, smaller access pathways are preferred. One key tissue sparing component to the procedure is that of access through the nerve space into the joint. The access pathway for an interbody fusion device must traditionally be large enough to accommodate the device; therefore, smaller implants are preferred. Further, it is preferable to have all surgical activities performed via a single access route. This may help reduce the amount of nerve and other tissues affected by the procedure.

Further, the size of the interbody fusion device can have a large impact on the success of the procedure. The size of the interbody fusion device should be large enough to support the high loads the implant will see during normal activities of the patient, while being small enough to minimize the surgical impact to surrounding tissues, as described above. Another key balance point is that of the cross-sectional area of the interbody fusion implant, versus that of the cavity in which bone graft is inserted into the interbody space. Essentially, more bone graft improves healing, while more implant structure helps support the joint mechanically before the healing is completed. The need to balance these factors has proved a challenge in the design of prior art interbody fusion devices, with many devices failing to provide short-term structural support and/or long-term bony in-growth and healing.

### SUMMARY

The various systems and methods of the present technology have been developed in response to the present state of the art, and in particular, in response to the problems and needs in the art that have not yet been fully solved by currently available suture anchoring systems and methods.

According to one embodiment, an interbody fusion system may include an interbody fusion device implantable in an interbody space of a spinal joint. The interbody fusion device may include a first component and a second component. The first component and the second component, together, may define a mating interface at which the first component and the second component are securable together with the first component and the second component both positioned entirely within the interbody space. The first component and the second component, together, may have at least a first cavity that is exposed on a superior side and on an inferior side of the interbody fusion device.

The first component may have a first component first lateral end and a first component second lateral end. The second component may have a second component first lateral end and a second component second lateral end. The first component first lateral end and the second component second lateral end may define at least part of the mating interface.

The mating interface may secure the first component and the second component together via relative inferior-superior motion of the first and second components within the interbody space.

The first component first lateral end may have a first component lip that extends superiorly to define a first component groove. The second component second lateral end may have a second component lip that extends inferiorly to define a second component groove. The first component lip may be insertable into the second component groove such that the first component lip frictionally engages the second component groove. The second component lip may be insertable into the first component groove such that the second component lip frictionally engages the first component groove.

The mating interface further comprises a locking member positionable in a locked configuration in which the locking member locks the first component and the second component together.

The locking member may engage the first component and/or the second component such that the locking member is movable between the locked configuration and an unlocked configuration, in which the first and second components are not secured together by the locking member.

The locking member may be rotatable, relative to the first and second components, from the unlocked configuration to the locked configuration.

The first component may further have a first component proximal end. The second component may further have a second component proximal end. The first component proximal end and the second component proximal end may be positionable adjacent to each other to define a proximal aperture sized to receive the locking member.

The locking member may have a generally cylindrical shape with a locking member proximal end that engages the proximal aperture in the locked configuration and the unlocked configuration.

The first component may further have a first component distal end. The second component may further have a second component distal end. The first component distal end and the second component distal end may be positionable adjacent to each other to define a distal aperture sized to receive a locking member distal end of the locking member.

The locking member may have an intermediate portion with a plurality of superior teeth and a plurality of inferior teeth. In the locked configuration, the superior teeth may be oriented superiorly and the inferior teeth may be oriented inferiorly.

The first component may have the first cavity. The second component may have a second cavity that is exposed on the superior side and on the inferior side of the interbody fusion device.

The first component may have a first component proximal end with a first component aperture through which material is insertable into the first cavity with the first component in the interbody space. The second component may have a second component proximal end with a second component aperture through which material is insertable into the second cavity with the second component in the interbody space.

The system may further include a first inserter with a first bore. The first inserter may be securable to the first component proximal end with the first bore aligned with the first component aperture to permit delivery of the material from the first bore through the first component aperture.

The system may further include a second inserter with a second bore. The second inserter may be securable to the second component proximal end with the second bore aligned with the second component aperture to permit delivery of the material from the second bore through the second component aperture.

The mating interface may be configured to permit the first and second components to be secured together without requiring anterior-posterior translation of the second component relative to the first component.

One method for implanting an interbody fusion device in an interbody space of a spinal joint may include inserting a first component of the interbody fusion device into the interbody space, inserting a second component of the interbody fusion device into the interbody space, and, with the first and second components positioned entirely within the interbody space, using a mating interface defined by the first and second components to secure the first component and the second component together. The first component and the second component, together, may have at least a first cavity that is exposed on a superior side and on an inferior side of the interbody fusion device.

The first component may have a first component first lateral end and a first component second lateral end. The second component may have a second component first lateral end and a second component second lateral end. The first component first lateral end and the second component second lateral end may define at least part of the mating interface. Using the mating interface to secure the first component and the second component together may include securing the first component first lateral end to the second component second lateral end.

Securing the first component first lateral end to the second component second lateral end may include moving the second component inferiorly or superiorly relative to the first component to cause the first component first lateral end to engage the second component second lateral end.

The first component first lateral end may include a first component lip that extends superiorly to define a first component groove. The second component second lateral end may include a second component lip that extends inferiorly to define a second component groove. Moving the second component inferiorly or superiorly relative to the first component to cause the first component first lateral end to engage the second component second lateral end may include inserting the first component lip into the second component groove such that the first component lip frictionally engages the second component groove, and inserting the second component lip into the first component groove such that the second component lip frictionally engages the first component groove.

The mating interface may further include a locking member. The method may further include positioning the locking member in a locked configuration, relative to the first and second components, in which the locking member locks the first component and the second component together.

The method may further include, prior to positioning the locking member in the locked configuration, engaging the first component and/or the second component with the locking member such that the locking member is in an unlocked configuration, relative to the first and second components, in which the first and second components are not secured together by the locking member.

Positioning the locking member in the locked configuration may include rotating the locking member, relative to the first and second components, from the unlocked configuration to the locked configuration.

The first component may further have a first component proximal end. The second component may further have a second component proximal end. The method further include, prior to positioning the locking member in the locked configuration, positioning the first component proximal end and the second component proximal end adjacent to each other to define a proximal aperture, and engaging the first component and/or the second component with the locking member comprises inserting the locking member into the proximal aperture.

The locking member may have a generally cylindrical shape with a locking member proximal end. Engaging the first component and/or the second component with the locking member may further include engaging the proximal aperture with the locking member proximal end.

The first component may further have a first component distal end. The second component may further have a second component distal end. The method may further include, prior to positioning the locking member in the locked configuration, positioning the first component distal end and the second component distal end adjacent to each other to define a distal aperture, and engaging the first component and/or the second component with the locking member comprises inserting a locking member distal end of the locking member into the distal aperture.

The locking member may have an intermediate portion with a plurality of superior teeth and a plurality of inferior teeth. Positioning the locking member in the locked configuration may include orienting the superior teeth are superiorly and orienting the inferior teeth inferiorly.

The first component may include the first cavity. The second component may include a second cavity that is exposed on the superior side and on the inferior side of the interbody fusion device. The method may further include positioning a material selected to promote bone in-growth in the first cavity and the second cavity.

The first component may have a first component proximal end with a first component aperture. The second component may have a second component proximal end with a second component aperture. The method may further include, with the first component in the interbody space, inserting the material into the first cavity through the first component aperture, and, with the second component in the interbody space, inserting the material into the second cavity through the second component aperture.

The method may further include securing a first inserter with a first bore to the first component proximal end. Inserting the material into the first cavity through the first component aperture may include delivering the material from the first bore through the first component aperture.

The method may further include securing a second inserter with a second bore to the second component proximal end. Inserting the material into the second cavity through the second component aperture may include delivering the material from the second bore through the second component aperture.

Using the mating interface to secure the first component and the second component together may include securing the first component and the second component together independently of anterior-posterior translation of the second component relative to the first component.

The method may include, prior to inserting the first component into the interbody space, securing a first proximal end of the first component to a first inserter, and, prior to inserting the second component into the interbody space, securing a second proximal end of the second component to a second inserter. Using the mating interface to secure the first component and the second component together may include, with the first component and the second component positioned entirely within the interbody space, exerting force on the first inserter and the second inserter such that the first inserter and the second inserter urge the first component and the second component to move relative to each other in a manner that secures the first component to the second component.

The method may further include, prior to inserting the first component into the interbody space, securing a first proximal end of the first component to a first inserter, prior to inserting the second component into the interbody space, securing a second proximal end of the second component to a second inserter, after securing the first component and the second component together, detaching the first inserter from the first component, and, after detaching the first inserter from the first component, exerting force on the second inserter to reposition the first component and the second component within the interbody space.

Inserting the second component into the interbody space may include inserting the second component into the interbody space after insertion of the first component into the interbody space.

These and other features and advantages of the present technology will become more fully apparent from the following description and appended claims, or may be learned by the practice of the technology as set forth hereinafter.

### BRIEF DESCRIPTION OF THE DRAWINGS

Various embodiments of the technology will be described in conjunction with the accompanying drawings. These drawings are to be construed as non-limiting examples of the present technology; those of skill in the art will appreciate that a wide variety of modifications are possible within the scope and spirit of the technology.

FIG. 1 is a perspective view depicting the transverse-lateral insertion of an interbody fusion device, according to one embodiment.

FIG. 2 depicts an interbody fusion device according to another embodiment.

FIGS. 3A and 3B depict posterior elevation and side elevation views, respectively, of a system including an interbody fusion device and rods retained by pedicle screws anchored in the pedicles of the vertebrae.

FIG. 4 depicts a transforaminal approach, according to one embodiment.

FIGS. 5, 6, and 7 are perspective, front elevation, and plan views, respectively, of an interbody fusion device and insertion instrumentation according to one embodiment.

FIGS. 8, 9, 10, and 11 are exploded, assembled, unlocked, and locked views, respectively, of an interbody fusion device according to one alternative embodiment.

### DETAILED DESCRIPTION

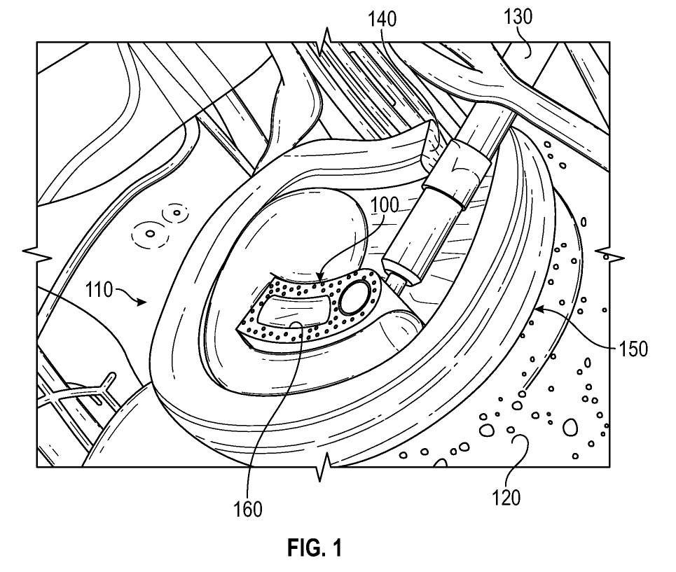

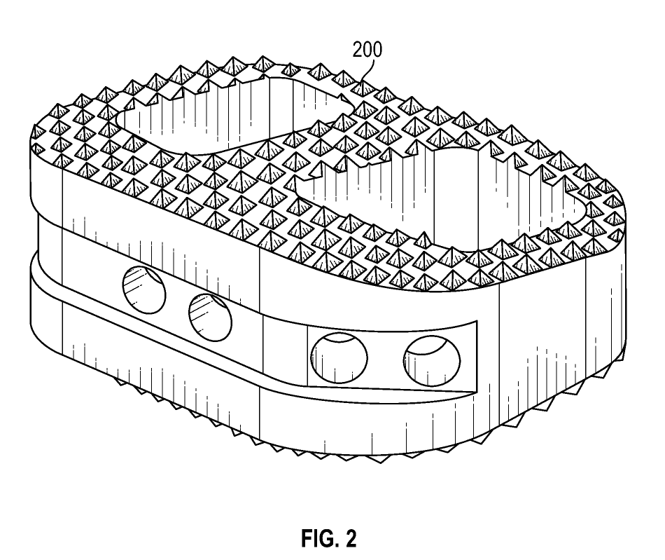

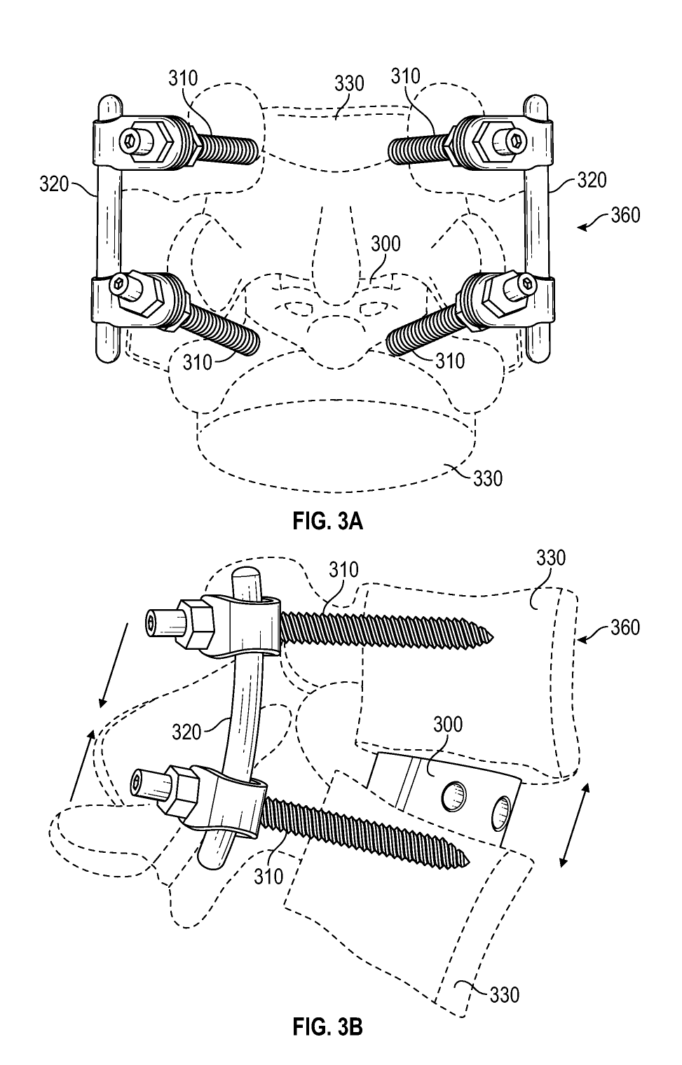

Exemplary embodiments will be best understood by reference to the drawings, wherein like parts are designated by like numerals throughout. It will be readily understood that the components described and illustrated in the Figures herein, could be arranged and designed in a wide variety of different configurations. Thus, the following more detailed description of the embodiments of the apparatus, system, and method, as represented in FIGS. 1 through 11, is not intended to limit the scope of the present disclosure, as claimed in this or any other application claiming priority to this application, but is merely representative exemplary of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

An “interbody fusion device” is any device designed to promote fusion between two different vertebral bodies, which may be at adjacent or non-adjacent levels of a spine. A “mating interface” includes the features of two components that facilitate a process by which the two components are mated together. A “cavity” is a hollow space within an object, which may be accessible through one or more openings in the object.

The term “proximal” relates to an object or feature that, in surgical use, is relatively closer to a surgeon or other medical practitioner. Conversely, the term “distal” relates to an object or feature that, in surgical use, is relatively further from the surgeon or other medical practitioner.

The term “lateral” relates to an object or feature that is relatively closer to the sides of a patient's body. Conversely, the term “medial” relates to an object or feature that is relatively closer to a “sagittal plane” of the patient's body, which is the plane that defines the boundary between the left and right halves of the patient's body. A “medial-lateral direction,” and variants thereof, relate to motion or geometry that extends generally perpendicular to the sagittal plane.

The term “cephalad” relates to an object or feature that is relatively closer to the top of the patient's head. Conversely, the term “caudal” relates to an object or feature that is relatively closer to the soles of the patient's feet. The phrases “cephalad-caudal direction,” “superior-inferior direction,” and variants thereof relate to motion or geometry that extends generally parallel to the sagittal plane and to a “coronal plane,” which is the plane that defines the boundary between the front and rear halves of the patient's body.

The term “anterior” relates to an object or feature that is relatively closer to the front of the patient's body. Conversely, “posterior” and “dorsal” relate to an object or feature that is relatively closer to the rear of the patient's body. The phrase “anterior-posterior direction” and variants thereof relate to motion or geometry that extends generally perpendicular to the coronal plane.

The present disclosure provides interbody fusion devices and instruments that help provide solid structural support to a joint to be fused, while also minimizing the size of the required surgical access pathway. The embodiments shown and described below generally utilize a transverse-lateral approach; however, those of skill in the art will recognize that the implants, instruments, and principles set forth herein may readily be adapted to other approaches for interbody fusion. Further, these embodiments are shown in the context of lumbar interbody fusion, for example, for the L4-L5 joint and the L5-S1 joint; however, those of skill in the art will further recognize how they may be adapted for other spinal joints. In some embodiments, a transforaminal, transverse-lateral approach may be used. This approach may provide improved access with minimal tissue disruption.

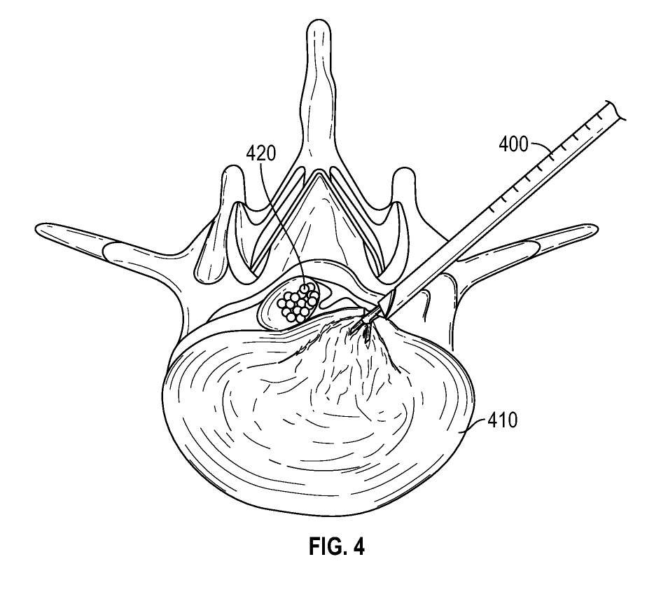

FIG. 4 depicts a transforaminal approach, according to one embodiment. An instrument 400 may be inserted into an interbody space 410, through the posterior region immediately lateral to the nerve bundles 420 in the for aminal space.

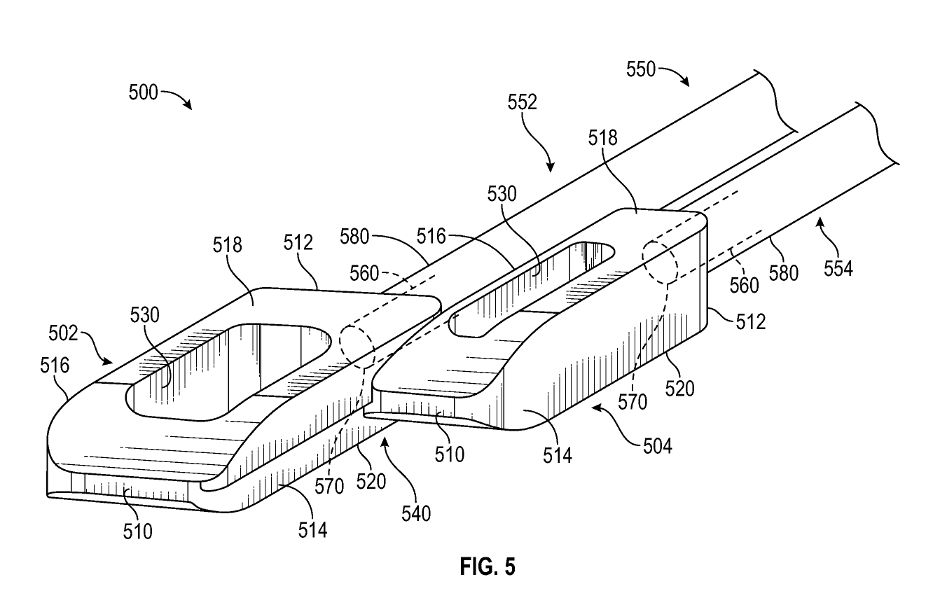

In some embodiments, the benefits of a large footprint and narrow access pathway may both be obtained through the use of a modular interbody fusion device. The components of the modular interbody fusion device may be inserted into the joint space sequentially, with each successive component mating with those that have been previously inserted. The components may thus be rigidly secured together to provide the benefits of a single interbody fusion device with a much larger footprint, without requiring a larger access pathway. One such embodiment will be shown and described in connection with FIGS. 5, 6, and 7.

FIGS. 5, 6, and 7 are perspective, front elevation, and plan views, respectively, of an interbody fusion device, or device 500, and insertion instrumentation 550 according to one embodiment. As shown in FIG. 5, the device 500 may include multiple components that can be inserted sequentially into an interbody space (such as the interbody space 110 of FIG. 1) sequentially. The device 500 may include a first component 502 and a second component 504. The insertion instrumentation 550 may include a first inserter 552 coupled to the first component 502 for insertion, and a second inserter 554 coupled to the second component 504 for insertion.

The first component 502 and the second component 504 of the device 500 may each have a leading end 510, a trailing end 512, a first lateral end 514, a second lateral end 516, a superior end 518, and an inferior end 520. As shown, the leading ends 510 may be tapered to facilitate motion of the first component 502 and the second component 504 through the tissues remaining in the interbody space 110. If desired, some tissues, such as some or all of the natural nucleus of the joint, may be extracted from the interbody space 110 prior to insertion of the device 500. Other ends and/or corners of the first component 502 and the second component 504 may also be rounded so as to help prevent disrupting surrounding tissues during insertion and/or during the healing process.

The first component 502 and the second component 504 may each have a cavity 530 that extends from the superior end 518 to the inferior end 520. Each cavity 530 may be sized to hold a quantity of bone graft, or one or more other bony ingrowth-enhancing substances. Each cavity 530 may be open to the superior end 518 and the inferior end 520 of the component such that a bony column can form between the two vertebral body end plates facing the device 500.

The first component 502 and the second component 504 may be made to be attachable together within the interbody space 110. This may be accomplished through the use of a mating interface 540 between the first lateral end 514 of the first component 502 and the second lateral end 516 of the second component 504. According to some methods, the first component 502 may first be inserted into the interbody space 110. After the first component 502 has been inserted, the second component 504 may be inserted alongside the first component 502 and secured to the first component 502. Optionally, the first component 502 may be moved, for example, medially, within the interbody space 110 prior to insertion of the second component 504.

The mating interface 540 may be configured in various ways. In some embodiments, the mating interface 540 may be configured such that the second component 504 can slide into engagement with the first component 502 during insertion. Additionally or alternatively, the mating interface 540 may be configured such that the first component 502 and the second component 504 can be moved in the cephalad/caudal direction relative to each other in order to cause the second component 504 to come into engagement with the first component 502.

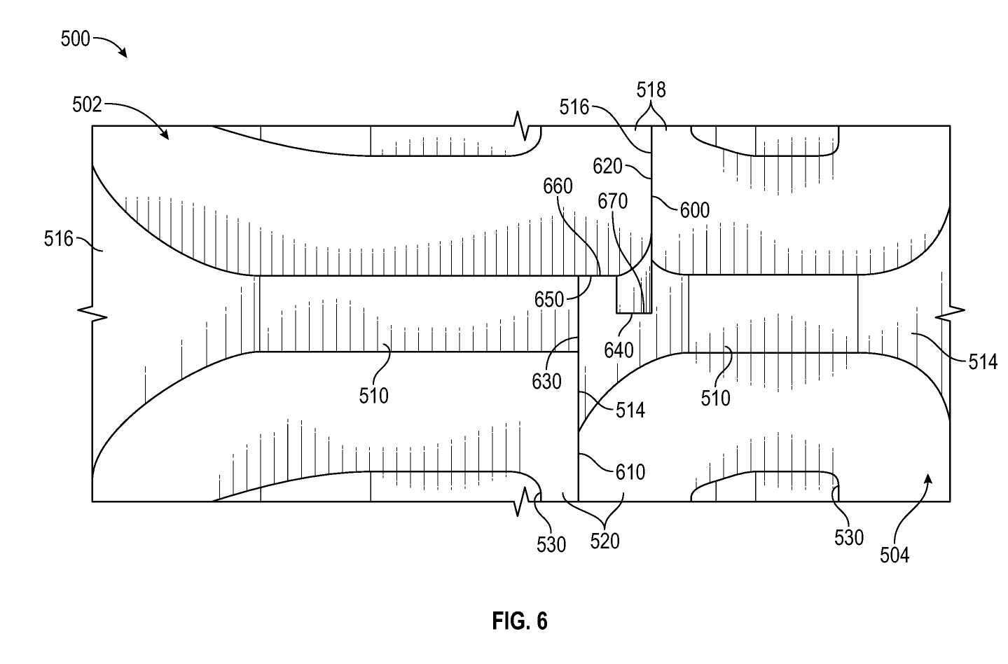

As more clearly shown in FIG. 6, the mating interface 540 may be defined by the relative shapes of the first lateral end 514 of the first component 502 and the second lateral end 516 of the second component 504. Specifically, the first lateral end 514 of the first component 502 may have a superior portion 600 and an inferior portion 610. Similarly, the second lateral end 516 of the second component 504 may have a superior portion 620 and an inferior portion 630. When the device 500 is fully assembled, the superior portion 600 may lie alongside the superior portion 620, and the inferior portion 610 may lie alongside the inferior portion 630. The superior portion 600 may protrude beyond the inferior portion 610, and the inferior portion 630 may protrude beyond the superior portion 620, as depicted in FIG. 6.

Further, the mating interface 540 may include a lip 640 extending inferiorly from the superior portion 600 of the first component 502, and a lip 660 extending superiorly from the inferior portion 630 of the second component 504. The lip 640 may be spaced apart from the inferior portion 610 of the first component 502 by a groove 650. Similarly, the lip 660 may be spaced apart from the superior portion 620 of the second component 504 by a groove 670. The lip 660 may have a width selected to allow the lip 660 to fit into the groove 650 of the first component 502. Similarly, the lip 640 may have a width selected to allow the lip 640 to fit into the groove 670 of the second component 504.

If desired, the lip 640 and the lip 660 may be sized to fit into the groove 670 and the groove 650 with some interference so that, once the first component 502 and the second component 504 have been coupled together, the friction involved in the interlocking of the lip 640 and the lip 660 with each other tends to keep the first component 502 in place relative to the second component 504.

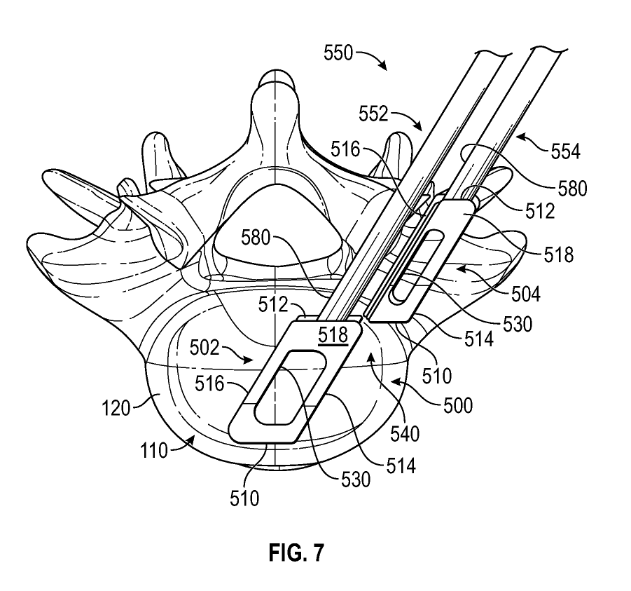

As depicted in FIG. 7, the first component 502 and the second component 504 may be inserted one-at-a-time into the interbody space 110. In some embodiments, the first component 502 may be inserted first, via a transforaminal, postero-lateral approach, as described previously. The first component 502 may first be secured to a distal end 580 of the first inserter 552, and then the proximal end (not shown) of the first inserter 552 may be grasped and manipulated to push the first component 502 through the access pathway, which may be opened and maintained through the use of retractors, cannulas, and/or other devices known in the art. The first inserter 552 may be coupled to the trailing end 512 of the first component 502 via various clips, clasps, and/or fittings known in the art; in one embodiment, the distal end 580 of the first inserter 552 may be coupled to the trailing end 512 of the first component 502 via a bayonet fitting or the like. The proximal end (not shown) of the first inserter 552 may have a handle or other user interface that facilitates manual manipulation. The second inserter 554 may optionally have a configuration similar to that of the first inserter 552.

If desired, the first inserter 552 may be used to urge the first component 502 to move medially prior to insertion of the second component 504. Thus, the first inserter 552 may be moved to the medial side of the access pathway to make more space for the second component 504 and the second inserter 554. Additionally, it may be desirable to move the first component 502 medially within the interbody space 110 so that the device 500, in its fully assembled state, is positioned proximate the center of the interbody space 110.

The second component 504 may then be secured to the distal end 580 of the second inserter 554 and inserted through the access pathway, as shown in FIG. 7. The proximal end (not shown) of the second inserter 554 may be grasped and manipulated to urge the second component 504 through the access pathway.

If desired, a brace (not shown) may be included in the insertion instrumentation 550, and may slidably couple the first inserter 552 to the second inserter 554 in a manner that constrains motion of the second inserter 554 relative to the first inserter 552, such that the second component 504 slides into proper position next to the first component 502. Such a brace may optionally have features that engage the first inserter 552 and the second inserter 554 to prevent relative rotation of each of the first inserter 552 and the second inserter 554 about its corresponding axis, thereby ensuring that the second component 504 is at a proper orientation, relative to the first component 502, to engage the first component 502 upon insertion into the interbody space 110. In such an embodiment, the mating interface 540 may function such that the lip 660 of the second component 504 enters the groove 650 of the first component 502 at or near the trailing end 512 of the first component 502, and the lip 640 of the first component 502 enters the groove 670 of the second component 504 at or near the leading end 510 of the second component 504.

In alternative embodiments, no such brace may be present, or a brace may be used that permits the second component 504 to be inserted into the interbody space 110 in such a manner that the second component 504 does not engage the first component 502 upon insertion into the interbody space 110. In such an embodiment, the first inserter 552 and/or the second inserter 554 may be manipulated such that the second component 504 moves superiorly, relative to the first component 502, within the interbody space 110. The lip 640 of the first component 502 may slide inferiorly against the superior portion 620 of the second lateral end 516 of the second component 504 until the lip 640 slides into the groove 670 of the second component 504. Similarly, the lip 660 of the second component 504 may slide superiorly against the inferior portion 610 of the first lateral end 514 of the first component 502 until the lip 660 slides into the groove 650 of the first component 502. Optionally, the lip 660 may have a tapered tip (not shown) pointing superiorly, and the lip 640 may have a tapered tip (not shown) pointing inferiorly to facilitate insertion of the lip 660 into the groove 650 and facilitate insertion of the lip 640 into the groove 670.

The ability to couple the first component 502 and the second component 504 together along the cephalad-caudal direction may make the second component 504 easier to insert into the interbody space 110, since the second component 504 need not frictionally engage the first component 502 as it moves into the interbody space 110. If desired, the insertion instrumentation 550 may include one or more instruments that facilitate the relative inferior/superior motion between the first component 502 and the second component 504 described above. For example, a clamping mechanism (not shown) may grip the first inserter 552 and the second inserter 554, proximate the distal ends 580 of the first inserter 552 and the second inserter 554, or at a location outside the body. The clamping mechanism may urge the first inserter 552 and the second inserter 554 into a parallel relative position to urge the lip 640 and the lip 660 to enter the groove 670 and the groove 650, respectively, as described above.

Such a clamping mechanism may have a worm gear or other device (not shown) that provides a mechanical advantage so that the physician can apply a lower level of force to the device, while obtaining a higher force that urges the first component 502 and the second component 504 into the generally coplanar configuration shown in FIG. 6. Thus, the lip 640, the 660, the groove 670, and the groove 650 may be relatively sized such that significant interference exists between these elements to keep the first component 502 and the second component 504 in such a coplanar configuration. The relatively high force needed to push the first component 502 and the second component 504 together in this manner may be provided by the clamping mechanism.

Optionally, the bone graft and/or other substance(s) may be packed into each cavity 530 prior to insertion of the corresponding component into the interbody space 110. Additionally or alternatively, the bone graft and/or other substance(s) may be inserted after insertion through the insertion instrumentation 550. For example, the first inserter 552 and the second inserter 554 may each be cannulated to permit passage of material therethrough, into the cavities 530 of the first component 502 and the second component 504. Specifically, the first inserter 552 and the second inserter 554 may each have a bore 560 (shown in FIG. 5) sized to permit passage of the materials.

Each bore 560 may be accessible through an aperture 570 (shown in FIG. 5) in the distal end 580 of the first inserter 552 and the second inserter 554, and through a corresponding aperture (not shown) in the proximal end of the first inserter 552 and the second inserter 554. Thus, material, such as bone graft and/or other substances, may be passed from the proximal ends of the first inserter 552 and the second inserter 554 into the cavities 530 of the first component 502 and the second component 504, with the first component 502 and the second component 504 disposed in the interbody space 110. Rams (not shown) or other instrument may be used to urge the bone graft and/or other substances through the bores 560 and into the cavities 530.

After the first component 502 and the second component 504 have been coupled together via the mating interface 540 to define the device 500, the device 500 may optionally be further repositioned within the interbody space 110. This repositioning may optionally be done after the bone graft and/or other substance(s) have been delivered to the cavities 530 of the first component 502 and the second component 504. According to one example, the first inserter 552 may be detached from the trailing end 512 of the first component 502, and the second inserter 554, alone, may be manipulated to position the device 500 at the desired location within the interbody space 110. Removal of the first inserter 552 may clear the access pathway, leaving room for the second inserter 554 to move medially within the access pathway to urge the device 500 to move medially within the interbody space 110.

As mentioned previously, many different mating interfaces may be used in connection with a modular interbody fusion device according to the present disclosure. The first lateral end 514 of the first component 502 and the second lateral end 516 of the second component 504 may be modified to incorporate any of a variety of mating interfaces known in the art. Furthermore, a modular interbody fusion device according to the present disclosure is not limited to two components; rather, in alternative embodiments (not shown), three or more components may be used. Each adjacent pair of components may be coupled together within the interbody space 110 through the use of a mating interface.

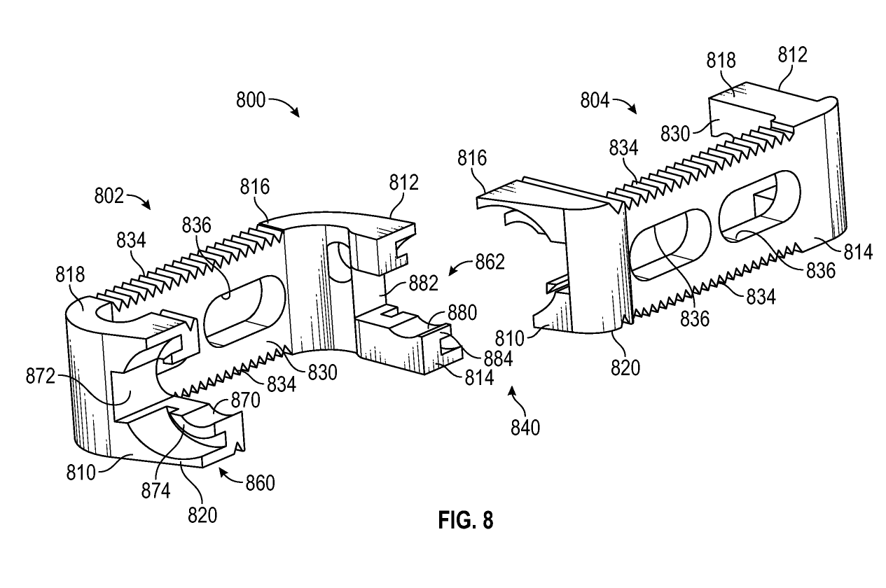

Further, many different modular interbody devices are envisioned within the scope of the present disclosure. One alternative example will be shown and described in connection with FIGS. 8 through 11.

FIGS. 8, 9, 10, and 11 are exploded, assembled, unlocked, and locked views, respectively, of an interbody fusion device, or device 800, according to one alternative embodiment. As shown in FIG. 8, the device 800 may include multiple components that can be inserted sequentially into an interbody space (such as the interbody space 110 of FIG. 1) sequentially. The device 800 may include a first component 802 and a second component 804.

The first component 502 and the second component 504 of the device 500 may each have a leading end 810, a trailing end 812, a first lateral end 814, a second lateral end 816, a superior end 818, and an inferior end 820. As shown, the corners of each of the first component 802 and the second component 804 may be rounded to facilitate motion of the first component 802 and the second component 804 through the tissues remaining in the interbody space 110. If desired, some tissues, such as some or all of the natural nucleus of the joint, may be extracted from the interbody space 110 prior to insertion of the device 800.

The first component 802 and the second component 804 may cooperate to define a cavity 830 that resides generally between the first component 802 and the second component 804. The cavity 830 may be sized to hold a quantity of bone graft, or one or more other bony ingrowth-enhancing substances. The cavity 830 may be open to the superior end 818 and the inferior end 820 of the component such that a bony column can form between the two vertebral body end plates facing the device 800.

The first component 802 and the second component 804 may be made to be attachable together within the interbody space 110. This may be accomplished through the use of a mating interface 840 between the first lateral end 814 of the first component 802 and the second lateral end 516 of the second component 504. According to some methods, the first component 802 may first be inserted into the interbody space 110. After the first component 802 has been inserted, the second component 804 may be inserted alongside the first component 802 and secured to the first component 802. Optionally, the first component 802 may be moved, for example, medially, within the interbody space 110 prior to insertion of the second component 804.

The superior end 818 and the inferior end 820 of each of the first component 802 and the second component 804 may each have teeth 834 oriented toward the end plates of the adjoining vertebral body end places to help the first component 802 and the second component 804 remain in place within the interbody space 110 once insertion of both components has been completed. The first lateral end 814 of the second component 804 and the second lateral end 816 of the first component 802 may have ports 836 formed therein to allow bony in-growth to occur laterally through the device 800.

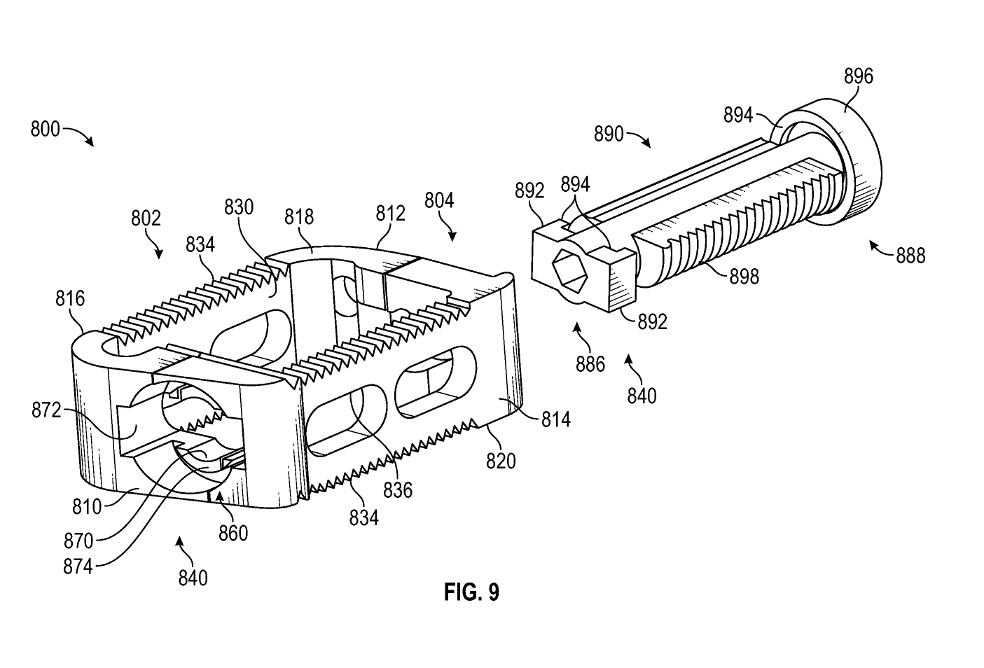

The mating interface 840 may be configured in various ways. As shown in FIG. 9, the mating interface 840 may be configured such that a locking member 850 is used to couple the first component 802 to the second component 804. Specifically, the second component 804 may be inserted alongside the second component 804 such that the first lateral end 814 of the first component 802 matches up with the second lateral end 816 of the second component 804.

The first lateral end 814 of the first component 802 may be complementarily shaped with the second lateral end 816 of the second component 804 such that these two surfaces cooperate to define a leading aperture 860 and a trailing aperture 862 (shown in FIG. 8). The leading aperture 860 may be generally keyhole-shaped, such that the leading aperture has a main portion 870 and two extensions 872 extending from the main portion 870. The leading aperture 860 may also have a retention ridge 874 that extends around the main portion 870 but is interrupted by the extensions 872. Similarly, the trailing aperture 862 may have a main portion 880, two extensions 882 extending from the main portion 880, and a retention ridge 884 that extends around the main portion 880 but is interrupted by the extensions 882.

The locking member 850 may be generally cylindrical, with a leading end 886, a trailing end 888, and an intermediate portion 890. The leading end 886 may have flanges 892 that extend to either side, with lips 894 protruding toward the trailing end 888. The trailing end 888 may have a cap 896 with a lip 894 that protrudes toward the leading end 886. Teeth 898 may protrude from the intermediate portion 890, in alignment with the flanges 892.

The locking member 850 may be shaped to be inserted through the trailing aperture 862 and through the leading aperture 860 when the first component 802 and the second component 804 are adjoining each other as shown in FIG. 9. The flanges 892 may be sized to fit through the extensions 882 of the trailing aperture 862 and then, after traversing the cavity 830, through the extensions 872 of the leading aperture 860. The cap 896 may seat in the trailing aperture 862 such that the lips 894 of the cap 896 engages the retention ridge 884 of the trailing aperture 862.

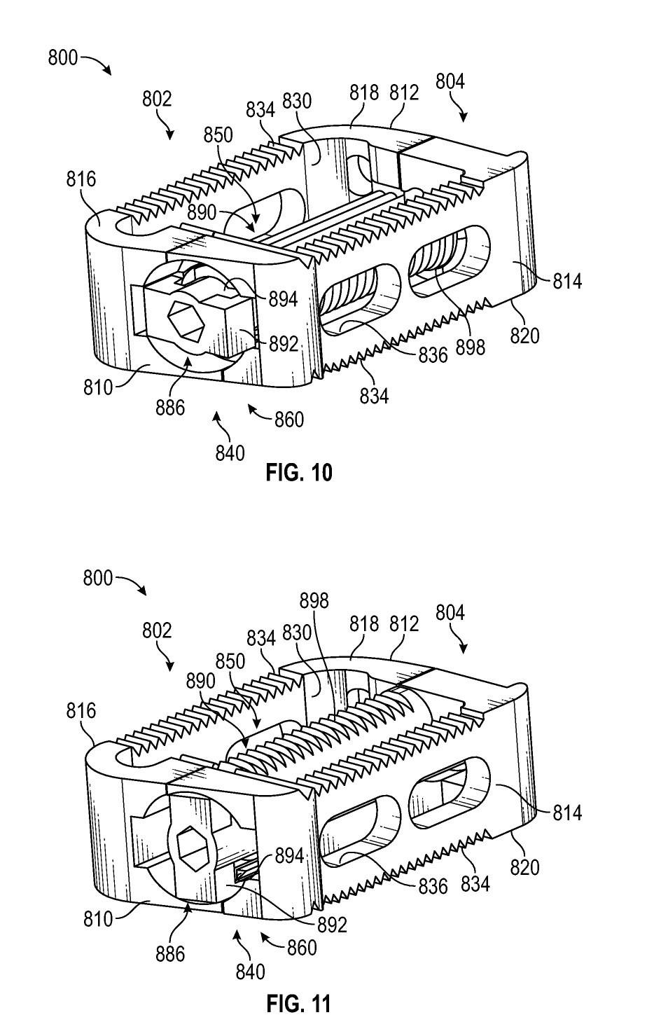

This is the configuration depicted in FIG. 10, which may be referred to as the “unlocked configuration,” in which the locking member 850 is not oriented to fully lock the first component 802 and the second component 804 together. Specifically, the lips 894 of the trailing end 888 of the locking member 850 may engage the retention ridge 884 of the trailing aperture 862 defined by the first component 802 and the second component 804 to keep the first component 802 and the second component 804 together at the trailing ends 812 thereof.

The teeth 898 may also be sized to fit through the extensions 882 of the trailing aperture 862. In the unlocked configuration, the teeth 898 may protrude medial-laterally, and may thus not engage the end plates of the adjacent vertebral bodies, as shown in FIG. 10.

FIG. 11 depicts the device 800 in the “locked configuration,” in which the locking member 850 is oriented to lock the first component 802 and the second component 804 together. The locking member 850 may be rotated 90° about its long axis to move from the unlocked configuration to the locked configuration. This may be done through the use of a tool, such as a hex driver (not shown), which may engage an interface, such as a hex socket (not shown) formed in the cap 896 of the trailing end 888 of the locking member 850. Such rotation of the locking member 850 may cause the lips 894 of the leading end 886 of the locking member 850 to rotate into engagement with the retention ridge 874 of the leading aperture 860 defined by the first component 802 and the second component 804. Such interlocking may keep the first component 802 and the second component 804 together at the leading ends 810 thereof so that, in combination with the interlocking of the lips 894 of the trailing end 888 of the locking member 850 with the retention ridge 884 of the trailing aperture 862, the first component 802 and the second component 804 are securely held together.

In the locked configuration, the teeth 898 may protrude toward the end plates of the vertebral bodies adjacent to the interbody space 110. The teeth 898 may be sized to engage the end plates in response to rotation of the locking member 850 to further lock the device 800 in position within the interbody space 110.

If desired, an instrument (not shown) may keep the first component 802 and the second component 804 together as the locking member 850 is inserted into the trailing aperture 862 and into the leading aperture 860. The same instrument may be used to drive the locking member 850 into position. In some examples, the instrument may have a distal interface (not shown) that grips the trailing ends 812 of the first component 802 and the second component 804 and keeps them in position; the instrument may further have a driver that carries the locking member 850 until it has been inserted and/or moved to the locked configuration. Such an instrument may also have a bore or other feature that conveys bone graft and/or other substance(s) into the cavity 830 when the first component 802 and the second component 804 are adjacent to each other, but the locking member 850 has not yet been inserted, as in FIG. 9. Such insertion may be similar to that described in connection with the device 500 of FIGS. 5, 6, and 7. Additionally or alternatively, the portion of the cavity 830 belonging to each of the first component 802 and the second component 804 may be packed with the bone graft and/or other substance(s) before they are inserted into the interbody space 110.

The device 500 and the device 800 may provide several benefits. Advantageously, the device 500 and the device 800 may each be assembled in-situ, or within the space between vertebral bodies. This may allow the surgeon to position both components of the device within the body, and then secure the components together in a separate step, rather than having to align the components together while one is inserted, so that it will mate with the other during insertion. The components may be secured together without requiring the application of excessive force, which could raise the risk of damage to the surrounding tissues.

Advantageously, the components need not be moved significantly relative to each other in the anterior-posterior direction in order to secure them together. Rather, the components may both be inserted along the anterior-posterior direction until they have both reached the desired position in the anterior-posterior direction. In the case of the device 500, the first component 502 and the second component 504 may then be moved together along the medial-lateral direction and/or the cephalad-caudal direction to secure them together. In the case of the device 800, the first component 802 and the second component 804 may be moved together within the intervertebral space, and then the locking member 850 may be used to secure the first component 802 and the second component 804 together. Notably, relative anterior-posterior motion of the first and second components may be carried out in the course of positioning the first and second components in the interbody space, before they have been secured together; however, the process of securing the first and second components together may not necessitate such relative anterior-posterior motion.

Further, the relative motion required in order to complete engagement between the components of the device may be relatively small. In the case of the device 500, relative cephalad-caudal motion between the first component 502 and the second component 504 along a relatively small distance may serve to mate the first component 502 with the second component 504. In the case of the device 800, the first component 802 and the second component 804 may be mated together without any relative motion between the first component 802 and the second component 804; rather, motion of the locking member 850 may be sufficient to mate the first component 802 and the second component 804 together.

Further, teeth for bone engagement may be provided at the center of the device, and not just at the edges of the device. The presence of such teeth may enhance the stability of the fused vertebral joint.

Yet further, the ability to deliver bone graft or other materials to the device in-situ may present benefits. Such delivery may facilitate implantation because the surgeon need not insert the materials into the device and keep the materials in place within the device during implantation.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the present disclosure. Embodiments in which an exclusive property or privilege is claimed are defined as follows.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

What is claimed is:

1. A system comprising:
an interbody fusion device implantable in an interbody space of a spinal joint, the interbody fusion device comprising:
a first component; and
a second component;
wherein the first component and the second component, together, define a mating interface at which the first component and the second component are securable together with the first component and the second component both positioned entirely within the interbody space;
wherein the first component and the second component, together, comprise at least a first cavity that is exposed on a superior side and on an inferior side of the interbody fusion device.

2. The system of claim 1, wherein:
the first component comprises a first component first lateral end and a first component second lateral end;
the second component comprises a second component first lateral end and a second component second lateral end;
the first component first lateral end and the second component second lateral end define at least part of the mating interface; and
the mating interface secures the first component and the second component together via relative inferior-superior motion of the first and second components within the interbody space.

3. The system of claim 2, wherein:
the first component first lateral end comprises a first component lip that extends superiorly to define a first component groove;
the second component second lateral end comprises a second component lip that extends inferiorly to define a second component groove;
the first component lip is insertable into the second component groove such that the first component lip frictionally engages the second component groove; and
the second component lip is insertable into the first component groove such that the second component lip frictionally engages the first component groove.

4. The system of claim 1, wherein:
the first component comprises a first component first lateral end and a first component second lateral end;
the second component comprises a second component first lateral end and a second component second lateral end;
the first component first lateral end and the second component second lateral end define at least part of the mating interface; and
the mating interface further comprises a locking member positionable in a locked configuration in which the locking member locks the first component and the second component together.

5. The system of claim 4, wherein the locking member engages the first component and/or the second component such that the locking member is movable between the locked configuration and an unlocked configuration, in which the first and second components are not secured together by the locking member.

6. The system of claim 5, wherein the locking member is rotatable, relative to the first and second components, from the unlocked configuration to the locked configuration.

7. The system of claim 5, wherein:
the first component further comprises a first component proximal end;
the second component further comprises a second component proximal end; and
the first component proximal end and the second component proximal end are positionable adjacent to each other to define a proximal aperture sized to receive the locking member.

8. The system of claim 7, wherein the locking member comprises a generally cylindrical shape with a locking member proximal end that engages the proximal aperture in the locked configuration and the unlocked configuration.

9. The system of claim 8, wherein:
the first component further comprises a first component distal end;
the second component further comprises a second component distal end; and
the first component distal end and the second component distal end are positionable adjacent to each other to define a distal aperture sized to receive a locking member distal end of the locking member.

10. The system of claim 9, wherein:
the locking member comprises an intermediate portion comprising a plurality of superior teeth and a plurality of inferior teeth; and
in the locked configuration, the superior teeth are oriented superiorly and the inferior teeth are oriented inferiorly.

11. The system of claim 1, wherein:
the first component comprises the first cavity;
the second component comprises a second cavity that is exposed on the superior side and on the inferior side of the interbody fusion device;
the first component comprises a first component proximal end comprising a first component aperture through which material is insertable into the first cavity with the first component in the interbody space; and
the second component comprises a second component proximal end comprising a second component aperture through which material is insertable into the second cavity with the second component in the interbody space.

12. The system of claim 11, further comprising a first inserter comprising a first bore;
wherein the first inserter is securable to the first component proximal end with the first bore aligned with the first component aperture to permit delivery of the material from the first bore through the first component aperture.

13. The system of claim 12, further comprising a second inserter comprising a second bore;
wherein the second inserter is securable to the second component proximal end with the second bore aligned with the second component aperture to permit delivery of the material from the second bore through the second component aperture.

14. The system of claim 1, wherein the mating interface is configured to permit the first and second components to be secured together without requiring anterior-posterior translation of the second component relative to the first component.

15. A method for implanting an interbody fusion device in an interbody space of a spinal joint, the method comprising:
inserting a first component of the interbody fusion device into the interbody space;
inserting a second component of the interbody fusion device into the interbody space; and
with the first and second components positioned entirely within the interbody space, using a mating interface defined by the first and second components to secure the first component and the second component together;
wherein the first component and the second component, together, comprise at least a first cavity that is exposed on a superior side and on an inferior side of the interbody fusion device.

16. The method of claim 15, wherein:
the first component comprises a first component first lateral end and a first component second lateral end;
the second component comprises a second component first lateral end and a second component second lateral end;
the first component first lateral end and the second component second lateral end define at least part of the mating interface;
using the mating interface to secure the first component and the second component together comprises securing the first component first lateral end to the second component second lateral end; and
securing the first component first lateral end to the second component second lateral end comprises moving the second component inferiorly or superiorly relative to the first component to cause the first component first lateral end to engage the second component second lateral end.

17. The method of claim 16, wherein:
the first component first lateral end comprises a first component lip that extends superiorly to define a first component groove;
the second component second lateral end comprises a second component lip that extends inferiorly to define a second component groove; and
moving the second component inferiorly or superiorly relative to the first component to cause the first component first lateral end to engage the second component second lateral end comprises:
inserting the first component lip into the second component groove such that the first component lip frictionally engages the second component groove; and
inserting the second component lip into the first component groove such that the second component lip frictionally engages the first component groove.

18. The method of claim 15, wherein:
the first component comprises a first component first lateral end and a first component second lateral end;
the second component comprises a second component first lateral end and a second component second lateral end;
the first component first lateral end and the second component second lateral end define at least part of the mating interface;
using the mating interface to secure the first component and the second component together comprises securing the first component first lateral end to the second component second lateral end;
the mating interface further comprises a locking member; and
the method further comprises positioning the locking member in a locked configuration, relative to the first and second components, in which the locking member locks the first component and the second component together.

19. The method of claim 18, wherein:
the method further comprises, prior to positioning the locking member in the locked configuration, engaging the first component and/or the second component with the locking member such that the locking member is in an unlocked configuration, relative to the first and second components, in which the first and second components are not secured together by the locking member; and
positioning the locking member in the locked configuration comprises rotating the locking member, relative to the first and second components, from the unlocked configuration to the locked configuration.

20. The method of claim 19, wherein:
the first component further comprises a first component proximal end and a first component distal end;
the second component further comprises a second component proximal end and a second component distal end;
the method further comprises, prior to positioning the locking member in the locked configuration:
positioning the first component proximal end and the second component proximal end adjacent to each other to define a proximal aperture; and
positioning the first component distal end and the second component distal end adjacent to each other to define a distal aperture; and
engaging the first component and/or the second component with the locking member comprises:
inserting the locking member into the proximal aperture; and
inserting a locking member distal end of the locking member into the distal aperture; and
engaging the proximal aperture with the locking member proximal end.

21. The method of claim 20, wherein:
the locking member comprises an intermediate portion comprising a plurality of superior teeth and a plurality of inferior teeth; and
positioning the locking member in the locked configuration comprises:
orienting the superior teeth are superiorly; and
orienting the inferior teeth inferiorly.

22. The method of claim 15, wherein:
the first component comprises the first cavity;
the second component comprises a second cavity that is exposed on the superior side and on the inferior side of the interbody fusion device;
the method further comprises positioning a material selected to promote bone in-growth in the first cavity and the second cavity;
the first component comprises a first component proximal end comprising a first component aperture;
the second component comprises a second component proximal end comprising a second component aperture; and
the method further comprises:
securing a first inserter comprising a first bore to the first component proximal end;
securing a second inserter comprising a second bore to the second component proximal end;
with the first component in the interbody space, delivering the material from the first bore through the first component aperture to the first cavity; and
with the second component in the interbody space, delivering the material from the second bore through the second component aperture to the second cavity.

23. The method of claim 15, wherein using the mating interface to secure the first component and the second component together comprises securing the first component and the second component together independently of anterior-posterior translation of the second component relative to the first component.

24. The method of claim 15, further comprising:
prior to inserting the first component into the interbody space, securing a first proximal end of the first component to a first inserter; and
prior to inserting the second component into the interbody space, securing a second proximal end of the second component to a second inserter;
wherein using the mating interface to secure the first component and the second component together comprises, with the first component and the second component positioned entirely within the interbody space, exerting force on the first inserter and the second inserter such that the first inserter and the second inserter urge the first component and the second component to move relative to each other in a manner that secures the first component to the second component.

25. The method of claim 15, further comprising:
prior to inserting the first component into the interbody space, securing a first proximal end of the first component to a first inserter;
prior to inserting the second component into the interbody space, securing a second proximal end of the second component to a second inserter;
after securing the first component and the second component together, detaching the first inserter from the first component; and
after detaching the first inserter from the first component, exerting force on the second inserter to reposition the first component and the second component within the interbody space;
wherein inserting the second component into the interbody space comprises inserting the second component into the interbody space after insertion of the first component into the interbody space.

26. A system comprising:
an interbody fusion device implantable in an interbody space of a spinal joint, the interbody fusion device comprising:
a first component shaped to define:
a first cavity that is exposed on a superior side and on an inferior side of the interbody fusion device; and
a first component proximal end comprising a first component aperture through which material is insertable into the first cavity with the first component in the interbody space;
a second component shaped to define:
a second cavity that is exposed on a superior side and on an inferior side of the interbody fusion device; and
a second component aperture through which material is insertable into the second cavity with the second component in the interbody space;
wherein the first component and the second component, together, define a mating interface at which the first component and the second component are securable together with the first component and the second component both positioned entirely within the interbody space;
wherein:
the first component comprises a first component first lateral end and a first component second lateral end;
the second component comprises a second component first lateral end and a second component second lateral end;
the first component first lateral end and the second component second lateral end define at least part of the mating interface; and
the mating interface is configured to permit the first and second components to be secured together without requiring anterior-posterior translation of the second component relative to the first component.
